(19) United States
(12) Patent Application Publication
(10) Pub. No.: US 2026/0355502 A1
(43) Pub. Date: Nov. 12, 2026

(54) INTERCHANGEABLE STIMULATION ASSEMBLY FOR WEARABLE NEUROSTIMULATION DEVICE

(71) Applicant: Encora, Inc., Boston, MA (US)

(72) Inventors: Daniel Carballo, Boston, MA (US); Allison Davanzo, Green Cove Springs, FL (US); Kyle Pina, Somerville, MA (US); Kristopher Vu, Malden, MA (US)

(21) Appl. No.: 19/746,273

(22) Filed: Jul. 17, 2026

Related U.S. Application Data
(63) Continuation of application No. 18/763,720, filed on Jul. 3, 2024.
(60) Provisional application No. 63/515,683, filed on Jul. 26, 2023.
(60) Provisional application No. 63/511,808, filed on Jul. 3, 2023.

(51) Int. Cl. A61N 1/04

(52) U.S. Cl. CPC A61N 1/0456; A61N 1/0484

(57) ABSTRACT

In an aspect, a wearable device for reducing symptoms of neurological movement disorders is presented. The wearable device includes controlling unit. The controlling unit includes a power supply and a controller in communication with the power supply. The controller is configured to generate stimulation output. The controlling unit includes an electrical connector in electrical communication with both the power supply and the controller. The wearable device includes an interchangeable stimulation assembly configured to provide the stimulation output to a body part of a patient through one or more stimulators within the interchangeable stimulation assembly positioned to be in contact with the body part. The interchangeable stimulation assembly includes a housing having an interior adapted to securely house both the power supply and the controller.

### CROSS REFERENCE TO RELATED APPLICATION

This application is a continuation of U.S. App. No. 18/763,720, filed Jul. 3, 2024, which claims priority to U.S. Provisional Application No. 63/511,808, filed Jul. 3, 2023, and U.S. Provisional Application No. 63/515,683, filed Jul. 26, 2023, the entirety of both of which is incorporated herein by reference.

### TECHNICAL FIELD

The present disclosure relates to wearable medical devices. In particular, the present disclosure relates to interchangeable stimulation assembly for wearable neurostimulation devices.

### SUMMARY

In an aspect, a wearable device for reducing symptoms of neurological movement disorders is presented. The wearable device includes a controlling unit. The controlling unit includes a power supply, a sensor, and a controller in communication with the power supply and the sensor. The controller is configured to generate stimulation output based on sensor data generated from the sensor. The controlling unit includes an electrical connector in electrical communication with both the power supply and the controller. The wearable device includes an interchangeable stimulation assembly configured to provide the stimulation output to a body part of a patient through one or more stimulators within the interchangeable stimulation assembly positioned to be in contact with the body part. The interchangeable stimulation assembly includes a housing having an interior adapted to securely house the controlling unit. The interchangeable stimulation assembly includes an identification unit. The interchangeable stimulation assembly includes an electrical connector port positioned within an interior of the housing and configured to connect with the electrical connector of the controlling unit. The controlling unit is configured to identify the interchangeable stimulation assembly via the identification unit. The controlling unit is configured to determine a performance metric of the one or more stimulators of the interchangeable stimulation assembly specific to the identity of the interchangeable simulation assembly. The controlling unit is configured to output, based on the performance metric, a system diagnostic results specific to the identity of the interchangeable stimulation assembly.

In another aspect, a method of tracking performance of a wearable device is presented. The method includes identifying, at a controlling unit connected to an interchangeable stimulation assembly via an electrical connector of the controlling unit, an identity of the interchangeable stimulation assembly via an identification unit of the interchangeable stimulation assembly. The controlling unit is configured to generate a stimulation output. The method includes determining a performance metric of one or more stimulators of the interchangeable stimulation assembly. The one or more stimulators are positioned within the interchangeable stimulation assembly to provide the stimulation output to a body part of a patient. The method includes outputting, based on the performance metric, a system diagnostic result of the interchangeable stimulation assembly specific to an identity of the interchangeable stimulation assembly.

The above and other preferred features, including various novel details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The disclosed embodiments have advantages and features which will be more readily apparent from the detailed description, the appended claims, and the accompanying figures(or drawings). A brief introduction of the figures is below.

FIG. 1 is a block diagram of a wearable neurostimulation device with an interchangeable stimulation assembly.

FIG. 2 is an illustration of an interchangeable stimulation assembly adjacent to a controlling unit.

FIG. 3 is an illustration of a bottom view of an interchangeable stimulation assembly adjacent to a controlling unit.

FIG. 4 is a zoomed-in illustration of an interchangeable stimulation assembly connecting to a controlling unit.

FIG. 5A-B illustrates top and bottom perspective views of an interchangeable stimulation assembly adjacent to a controlling unit.

FIG. 6 illustrates another embodiment of an interchangeable stimulation assembly adjacent to a controlling unit.

FIG. 7 illustrates a bottom perspective view of the illustration of FIG. 6;

FIG. 8 illustrates an embodiment of a wearable device;

FIG. 9 illustrates an interior of a controlling unit housed within an interchangeable stimulation assembly;

FIG. 10 illustrates a method of tracking performance of a wearable device;

FIG. 11 illustrates an embodiment of a wearable device;

FIG. 12 illustrates an exploded view of the wearable device shown in FIG. 11;

FIG. 13 is a flowchart illustrating an embodiment of a method of tracking performance of a wearable device;

FIG. 14 is a flowchart illustrating an embodiment of a method of tracking performance of a wearable device;

FIG. 15 is a flowchart illustrating an embodiment of a method of tracking performance of a wearable device.

### DETAILED DESCRIPTION

The Figures(FIGs.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

Aspects of the present disclosure may be used to provide for an interchangeability of one or more stimulators of wearable devices, such as wearable neurostimulation devices. In some embodiments, assemblies, systems, and methods herein may provide for increased durability of wearable neurostimulation devices, such as n increase in mechanical stress, moisture, temperature changes, repeated electrical or vibratory stimulation, and/or other parameters. Embodiments of the present disclosure may be used to provide for a low-profile electrical connector system that may enable long-lasting electrical connections between an interchangeable stimulation assembly and/or a control/power supply assembly.

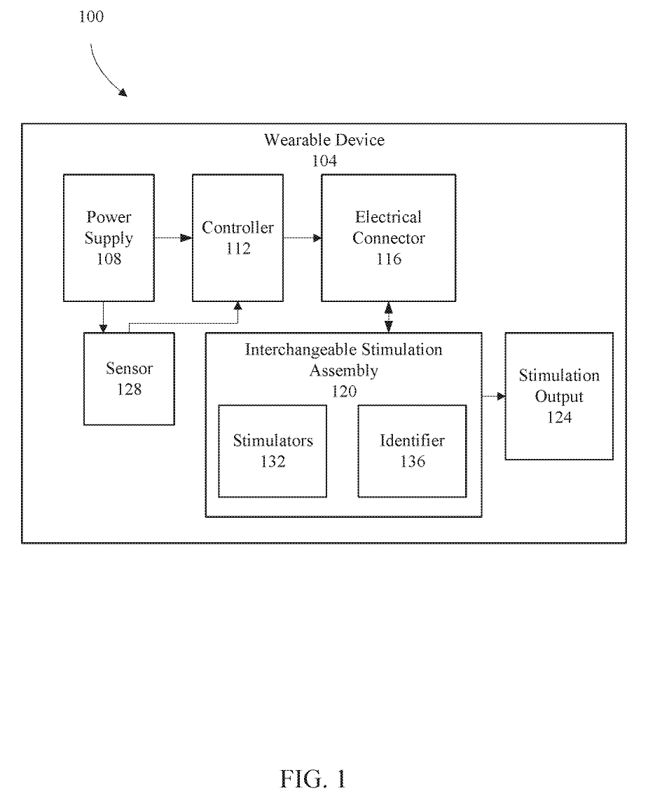

Referring now to FIG. 1, a block diagram 100 of a wearable neurostimulation device 104 with an interchangeable stimulation assembly 120 is presented. A “wearable device” as used in this disclosure refers to an object that is attachable or securable to at least a portion of a body part of a patient. For instance, wearable device 104 may be attachable to a patient via straps, locks, wristbands, and/or other attaching devices. Wearable device 104 may include a power supply 108. Power supply 108 may be a battery, such as, but not limited to, a lithium-ion battery. In some embodiments, power supply 108 may be rechargeable. Power supply 108 may be configured to provide voltage and/or current to one or more other components of wearable device 104, such as, but not limited to, controller 112, sensor 128, electrical connector 116, and/or interchangeable stimulation assembly 120 via electrical connector 116.

Wearable device 104 may include controller 112. Controller 112 may be any type of suitable processing device, such as, but not limited to, a microcontroller, microprocessor, system on a chip, ASIC, or other processing device. Controller 112 may be in electrical communication with power supply 108, electrical connector 116, and/or sensor 128. Sensor 128 may be any type of sensor, such as, but not limited to, an accelerometer, EKG sensor, inertial measurement unit (IMU), and/or other type of sensor. Sensor 128 may be configured to detect moment, electrical signals, or other data of a body part of a patient and generate sensor data. Sensor data may include, but is not limited to, voltages, currents, accelerations, EKG signals, positional data, and/or other types of data. For instance, sensor 128 may generate sensor data of muscle contractions of a body part of a patient. Body parts of a patient may include, but are not limited to, hands, arms, feet, legs, and/or other body parts. Controller 112 may be configured to receive sensor data of sensor 128 and generate stimulation output 124. Controller 112, power supply 108, and sensor 128 may be collectively referred to as a “controlling unit” throughout this disclosure.

A “stimulation output” as used in this disclosure refers to an output of one or more stimulators that stimulate one or more nerves of a body part of a patient. In some embodiments, controller 112 may be configured to determine optimal stimulation parameters for use in reducing symptoms of a movement disorder of a patient by using sensor-based optimization, including but not limited to model free reinforcement learning, genetic algorithms, and/or Q-learning. Parameters may include any quantities used to define a stimulation output such as, but not limited to, frequency, amplitude, phase, duty cycle, etc. In some embodiments, controller 112 may be configured to learn which parameters correspond to a highest reduction in symptom severity. Controller 112 may be configured to determine an optimal stimulation as a weighted average of optimal stimulations for each of independent symptoms detected in a patient by sensor 128. Weights may be proportional to a symptom severity relative to one or more other symptoms of a patient detected by sensor 128. As a non-limiting example, if a patient experienced tremors and rigidity, and the severity of the tremors was double that of the rigidity, the stimulation output would be two times the optimal tremor reducing pattern superposed with one times the optimal rigidity reducing pattern. In some embodiments, controller 112 may be configured to detect all active symptoms of a patient through sensor data generated by sensor 128 and may elect to reduce only the symptom with a worst severity. A “worst severity” as used in this disclosure refers to a symptom having a highest impact or detected value attributed to the symptom. For instance and without limitation, controller 112 may be configured to measure a shaking of a patient via sensor 128 and may assign a pattern of a stimulation output 124 that relates to a biggest decrease in a shaking amplitude of the patient.

Referring still to FIG. 1, controller 112 may be configured to compute a set of stimulation parameters based on raw or unfiltered data generated by sensor 128. Stimulation parameters of stimulation output 124 may be continuously updated in a closed feedback loop. Stimulation parameters can include any quantities used to define a stimulation waveform such as frequency, amplitude, phase, duty cycle, etc. At each iteration of an update in a closed feedback loop, current stimulation parameters and raw sensor input may be used to filter out stimulators 132 and/or sensor 128 crosstalk, either by using knowledge of stimulation output 124 to subtract from a sensed waveform or by using knowledge of the timing of the stimulation output 124 to limit sensing to the “off” phases of a pulsing stimulation. This filtering subsequently may allow for feature extraction of raw sensor input. Controller 112 may utilize a stimulation selection algorithm which may use current stimulation parameters and the extracted features to select new stimulation parameters. When the process repeats, the previously new stimulation parameters may become current stimulation parameters. A stimulation selection algorithm may include argmin (or minimization over the set of input arguments), Q-learning, neural networks, genetic algorithms, differential dynamic programming, iterative quadratic regulator, and/or guided policy, without limitation.

Controller 112 may be configured to extract one or more features of data generated by sensor 128. For instance controller 112 may input a filtered sensor signal, and may extract one or more temporal and/or spectral features. Examples of temporal features include, but are not limited to, the minimum value, the maximum value, first three standard deviation values, signal energy, root mean squared (RMS), zero crossing rate, principal component analysis (PCA), kernel or wavelet convolution, or autoconvolution. Examples of spectral features include, but are not limited to, the Fourier Transform, fundamental frequency, (Mel-frequency) Cepstral coefficients, the spectral centroid, and bandwidth. Features may be extracted with digital signal processing techniques via controller 112. A set of collected features may be fed into a stimulation selection algorithm via controller 112.

Wearable device 104 may include electrical connector 116. Electrical connector 116 may include one or more electrical wires, which may be made from copper, silver and/or other electrically conductive materials. Electrical connector 116 may be made of a durable material, such as rubber or other materials. Electrical connector 116 may be resilient to mechanical stress, moisture, temperature changes, stimulation outputs, and/or other factors. Electrical connector 116 may be electrically connected to controller 112 at a proximal side of electrical connector 116 relative to controller 112. Electrical connector 116 may include one or more connectors, such as, but not limited to, pogo pin connectors, magnetic connectors, zero insertion force (ZIF) connectors, flexible flat cable (FFC) connectors, flexible printed circuit board (FPC) connectors, micro-USB connectors and USB-C connectors, or a combination thereof. One or more connectors of electrical connector 116 may be adapted to connect electrical connector 116 to controller 112. For instance, controller 112 may be connected to electrical connector 116 via any one of the types of connectors described above, without limitation.

Electrical connector 116 may have a distal end relative to controller 112. A distal end of electrical connector 116 may be adapted to connect to interchangeable stimulation assembly 120. For instance and without limitation, electrical connector 116 may be adapted to connect to interchangeable stimulation assembly 120 through one or more of pogo pin connectors, magnetic connectors, ZIF connectors, FFC/FPC connectors, micro-USB connectors and USB-C connectors, or a combination thereof.

Still referring to FIG. 1, wearable device 104 may include interchangeable stimulation assembly 120. An “interchangeable stimulation assembly” as used throughout this disclosure refers to a stimulation device that is adapted to connect and disconnect from a wearable device. Interchangeable stimulation assembly 120 may be adapted to connect and/or disconnect from wearable device 104 via electrical connector 116. For instance, a patient may replace interchangeable stimulation assembly 120 with a new interchangeable stimulation assembly through operation of electrical connector 116. Electrical connector 116 may include one or more disconnection devices, such as, but not limited to, buttons, slidable locks, latches, or other disconnection devices. A patient may manually operate one or more disconnection devices of electrical connector 116, such as sliding a lock, pressing down on a depressible button, unhooking a latch, or other interactions. Interchangeable stimulation assembly 120 may include an interface adapted to couple to electrical connector 116. For instance and without limitation, interchangeable stimulation assembly 120 may include a power/data port that may be adapted to connect to electrical connector 116. In some embodiments, interchangeable stimulation assembly 120 may include on or more of pogo pin connectors, magnetic connectors, ZIF connectors, FFC/FPC connectors, micro-USB connectors and USB-C connectors, or a combination thereof. Electrical connector 116 may provide power from power supply 108 to interchangeable stimulation assembly 120.

Interchangeable stimulation assembly 120 may include a housing. For instance, a housing of interchangeable stimulation assembly 120 may be rectangular, square, circle, ovular, or other shapes. In some embodiments, interchangeable stimulation assembly 120 may have an interior of a housing. An interior of a housing of interchangeable stimulation assembly 120 may be hollow and may be adapted to receive controller 112 and/or power supply 108. A housing of interchangeable stimulation assembly 120 may be made of a flexible material that may allow for a stretching of one or more sides of the housing of interchangeable stimulation assembly 120 to conform to one or more sides of controller 112 and/or power supply 108. In some embodiments, power supply 108 and/or controller 112 may snap into a housing of interchangeable stimulation assembly 120. Interchangeable stimulation assembly 120 may have an electrical connector port. An electrical connector port may be positioned within a housing of interchangeable stimulation assembly 120. For instance and without limitation, an electrical connector port may be positioned within a right, left, front, or rear side of a housing of interchangeable stimulation assembly 120. In some embodiments, an electrical connector port of interchangeable stimulation assembly 120 may protrude from a surface of an interior of a housing of interchangeable stimulation assembly 120. An electrical connector port of interchangeable stimulation assembly 120 may protrude horizontally or vertically, relative to a housing of interchangeable stimulation assembly 120. An electrical connector port of interchangeable stimulation assembly 120 may be adapted to mate with or otherwise interface with electrical connector 116. For instance, an electrical connector port of interchangeable stimulation assembly 120 may be a male connector end that may be placed inside a female connector end of electrical connector 116.

Interchangeable stimulation assembly 120 may include one or more stimulators 132. A “stimulator” as used in this disclosure refers to a device that outputs a stimulating signal. Stimulators 132 may include, but are not limited to, electrodes, vibrational elements, ultrasonic emitters, and/or other types of stimulators. An electrical connector port of interchangeable stimulation assembly 120 may be in electrical connection with one or more stimulators 132 of interchangeable stimulation assembly 120. For instance, in some embodiments, an electrical cable may connect two or more stimulators 132 of interchangeable stimulation assembly 120 to an electrical connector port of interchangeable stimulation assembly 120, which may protrude from an interior surface of a housing of interchangeable stimulation assembly 120. Interchangeable stimulation assembly 120 may be replaceable by one or more other interchangeable stimulation assemblies 120. For instance, a first interchangeable stimulation assembly 120 may start to show physical signs of wear and/or stimulators 132 of interchangeable stimulation assembly 120 may start to have decreased performance. Interchangeable stimulation assembly 120 may be replaced with a new interchangeable stimulation assembly 120, such as by connecting controller 112, power supply 108, and/or sensor 128 to interchangeable stimulation assembly 120 through electrical connector 116.

Controller 112 may be configured to regulate and/or manage power delivered by power supply 108 to interchangeable stimulation assembly 120. In some embodiments, electrical connector 116 may provide a data connection between controller 112 and interchangeable stimulation assembly 120. A “data connection” used herein refers to a communicative coupling between two or more devices in which data is transmitted from one device to another. Data shared in a data connection provided by electrical connector 116 may include, but is not limited to, power data, sensor data, stimulation parameter data, software updates, firmware updates, stored data from a memory in communication with controller 112, and/or other data. For instance controller 112 may communicate power supply data with interchangeable stimulation assembly 120, such as, but not limited to, battery capacity, percent charged, battery health, and/or other information relating to power supply 108. Controller 112 may communicate stimulation parameter data. For instance, controller 112 may be configured to receive sensor data from sensor 128 and calculate one or more stimulation outputs 124, which may be communicated to and delivered by interchangeable stimulation assembly 120 to a body part of a patient.

Referring back to interchangeable stimulation assembly 120, in some embodiments, interchangeable stimulation assembly 120 may have two or more stimulators 132. Interchangeable stimulation assembly 120 may have a set of stimulators 132, such as a set of 2, 4, 6, 8, or more stimulators 132. In some embodiments, the actuators are resistive heating elements. In some embodiments, stimulators 132 may be vibration motors. In some embodiments, stimulators 132 may be electromagnets. In some embodiments, stimulators 132 may be electro permanent magnets. In some embodiments, stimulators 132 may be piezoelectric actuators. In some embodiments, stimulators 132 may be voice coil vibration motors. In some embodiments, stimulators 132 may be rotating eccentric mass vibration motors. Stimulators 132 may include one or more sonic transducers. Sonic transducers may include, without limitation, ultrasonic transducers, or other sonic transducers. Ultrasonic transducers may also be interchangeably referred to as “ultrasonic emitters” throughout this disclosure. Ultrasonic transducers in some embodiments may output a frequency of about 1 Hz to about 1 GHz. One or more ultrasonic transducers may be configured to target specific parts of a user’s body and/or at specific frequency ranges. For instance, one or more ultrasonic transducers may be configured to output a low-frequency ultrasound. A low-frequency ultrasound may include a frequency of about 20 kHz to about 100 kHz which may target larger nerve bundles, deep soft tissues, and/or joints of a user. A frequency of about 20 kHz to about 100 kHz may provide deep tissue penetration with less superficial heating.

One or more ultrasonic transducers may be configured to output a medium-frequency ultrasound. A medium-frequency ultrasound may include a frequency of about 100 kHz to about 500kHz. A frequency of about 100kHz to about 500 kHz may be used to target deeper nerves, muscles, tendons, and/or ligaments of a user. A frequency of about 100kHz to about 500 kHz may penetrate one or more tissues at intermediate depths and may affect structures like tendons and/or ligaments, may improve tissue perfusion, increase collagen extensibility, and potentially modulate nerve activity. In some embodiments, one or more ultrasonic transducers may be configured to output a high-frequency ultrasound. A high-frequency ultrasound may include a frequency of about 500kHz to about 1 MHz. A frequency of about 500 kHz to about 1 MHz may target superficial nerves, tendons, and/or ligaments of a user and may offer high resolution imaging of one or more parts of a user. A frequency of about 500 kHz to about 1 MHz may be used to target tissues closer to a skin surface of a user and may be used for localized treatments. In some embodiments, one or more ultrasonic transducers may be configured to output a diagnostic ultrasound. A diagnostic ultrasound may include a frequency of about 1 MHz to about 10 MHz. A frequency of about 1 MHz to about 10 MHz may provide imaging and visualization of soft tissues, organs, and/or superficial nerves. A frequency of about 1 MHz to about 10 MHz may provide for diagnostic imaging without therapeutic effects. In some embodiments, one or more ultrasonic transducers may be configured to output a very high-frequency ultrasound. A very high-frequency ultrasound may include a frequency of about or greater than 10 MHz. A frequency at about or greater than 10 MHz may target superficial structures such as skin layers and nerves close to a skin surface. A frequency at about or greater than 10 MHz may provide for ultra-high resolution imaging for dermatology, superficial nerve assessments, and/or visualization of superficial skin conditions. In an embodiment, two or more ultrasonic transducers may be used. Ultrasonic transducers may include piezoelectric, capacitive, or other transducers. In some embodiments, one or more ultrasonic transducers may be configured to apply a waveform output to a part of a user. A waveform output of one or more ultrasonic transducers may be about 1MHz in frequency.

With continued reference to FIG. 1, in some embodiments, stimulators 132 may be electrodes. Electrodes may be configured to output an electrical signal to a body part of a patient. For instance, and without limitation, electrodes may be configured to output an AC (alternating current), DC (direct current), or other signal to a user. One or more electrodes may be configured to output a waveform with a voltage of about 1V to about 5V, a current of about 10mA to about 50mA, and a frequency of about 50Mhz, without limitation. One or more electrodes may be configured to perform transcutaneous electrical nerve stimulations (TENS), functional electric stimulations (FES), and/or other stimulations. One or more electrodes may be configured to output amps in a range of about, but not limited to 1 mA to about 80 mA. In some embodiments, one or more electrodes may be configured to output a voltage of about 0V to about 50V. One or more electrodes may be configured to output a frequency of about 1Hz to about 100 Hz. In some embodiments, one or more electrodes may be configured to output a frequency of about 100 Hz to about 10,000 Hz. One or more electrodes may be configured to provide conventional TENS, high-frequency or burst TENS, and/or modulation TENS. One or more electrodes may be configured to output time-varying frequencies, square waveforms, pulse waveforms, and the like of one or more voltages, currents, and the like.

One or more electrodes may be configured to provide an intensity calibration of a stimulation for a user. As a non-limiting example, in TENS and/or FES intensity calibration, an intensity range of about 1 mA to about 5 mA may be used initially and may gradually increase until one or more parameters are met, such as if a patient feels a tingling or mild muscle twitching. Continuing this example, a current may be adjusted to about 10 mA to about 80 mA, depending on a treatment area and a patient’s comfort.

One or more electrodes may include shapes, sizes, dimensions, and the like. For instance, electrodes may include shapes such as, but not limited to, square, rectangular, round, butterfly, and the like. Electrodes may have a small size of about 2 cm by cm to about 5 cm by 5 cm. Electrodes may have a medium size of about 5 cm by 5 cm to about 10 cm by 5 cm. Electrodes may have a large size of about 10 cm by 5 cm or greater. Electrodes may include materials such as, but not limited to, carbon rubber electrodes, silver-based electrodes, and the like. Electrodes may have a resistance of about 1,000 ohms to about 5,000 ohms, without limitation. In some embodiments, electrodes may have a resistance less than 1,000 ohms or greater than 5,000 ohms, without limitation.

Interchangeable stimulation assembly 120 may be configured to provide simulation output 124 via one or more stimulators 132 of interchangeable stimulation assembly 120. Stimulation output 124 may be calculated by controller 112 as described above and may be communicated to interchangeable stimulation assembly 120. Interchangeable stimulation assembly 120 may provide stimulation output 124 to a body part of a patient based on data received by controller 112. One or more stimulators 132 of interchangeable stimulation assembly 120 may be located to contact one or more proprioceptive nerves of a body part of a patient. A “proprioceptive nerve” as used in this disclosure refers to a sensory nerve ending in the human body that provides the brain with information related to movement. Proprioceptive nerves may be found in proprioceptive tissue, such as, but not limited to, flexor carpi radialis, flexor carpi ulnaris, extensor carpi radialis, extensor carpi ulnaris, or a combination thereof. For instance and without limitation, one or more stimulators 132 of interchangeable stimulation assembly 120 may be positioned to contact and/or provide stimulation output 124 to one or more proprioceptive nerves in a wrist of a patient.

Referring still to FIG. 1, controller 112 may be configured to generate and/or track a life cycle of interchangeable stimulation assembly 120. A “life cycle” as used in this disclosure refers to the degradation and/or reduced capacity of performance of a device over time. Controller 112 may be configured to track various performance metrics of interchangeable stimulation assembly 120. A “performance metric” as used in this disclosure refers to a value of an operation of a device that can be compared to an ideal value of the operation of the device. In some embodiments, controller 112 may be configured to track various usage metrics of interchangeable stimulation assembly 120. A “usage metric” as used throughout this disclosure refers to data pertaining to quantities of outputs of a device over a period of time. such as, but not limited to, total operational time, simulation output 124 duration, frequency of use, interchangeable stimulation assembly 120 uptime, number of cycles of stimulation output 124, and/or a number of pulses provided by stimulation output 124. Controller 112 may compare one or more metrics of interchangeable stimulation assembly 120 to reference data that may be provided by input from a user and/or an external computing device. In some embodiments, controller 112 may automatically calculate initial metrics of interchangeable stimulation assembly 120 upon connection of a new interchangeable stimulation assembly 120 to wearable device 104 via electrical connector 116. Performance, usage, and/or other metrics of interchangeable stimulation assembly 120 may be used during a system diagnostic check performed by controller 112. A “system diagnostic check” as used throughout this disclosure refers to a comparison of one or more metrics of a device to ideal or operational values. A system diagnostic check may output a system diagnostic result. A system diagnostic result may be an indication of an operability of one or more stimulators 132 of interchangeable stimulation assembly 120. For instance, a system diagnostic result may indicate that interchangeable stimulation assembly 120 may be nearing an end of its lifetime, that a patient should replace interchangeable stimulation assembly 120 with a new interchangeable stimulation assembly 120, that firmware of interchangeable simulation assembly 120 may need to be updated, and/or other indications of operation of interchangeable stimulation assembly 120.

In some embodiments, metrics of interchangeable stimulation assembly 120 may be tracked via one or more sensors of interchangeable stimulation assembly 120 and/or of a controlling unit. For instance, sensors of interchangeable stimulation assembly 120 may include optical encoders or magnetic sensors, which may track a number of rotations or cycles of a motor of stimulators 132. A sensor of interchangeable stimulation assembly 120 may include a temperature sensor, which may be used to monitor operating temperature of interchangeable stimulation assembly 120. Controller 112 may be in communication with a sensing circuit, such as a current sensing circuit, which may be used to directly monitor voltages/currents of interchangeable stimulation assembly 120. Controller 112 may be configured to detect back EMF through a back EMF detection circuit, an impedance through an impedance measurement circuit, and/or other electrical parameters with corresponding electrical circuits. For instance, a current sensing circuit may include a low-resistance shunt resistor and a high-side current sense amplifier, which may measure an amount of current drawn by stimulators 132 during operation. A voltage drop across a shunt resistor, which may be proportional to a current flow, may be amplified and measured by an analog-to-digital converter (ADC). Controller 112 may calculate a current draw based on a known resistance of a shunt resistor.

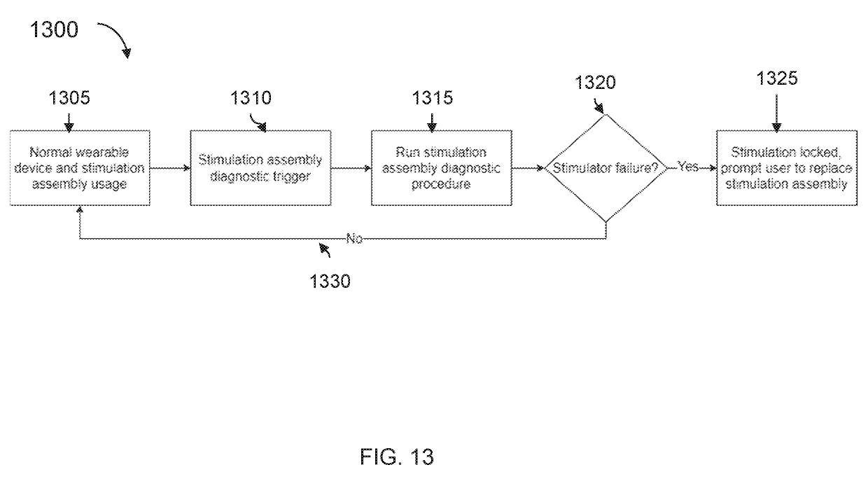

A back EMF circuit may be implemented using a voltage divider and an ADC. A voltage divider may be connected across terminals of one or more stimulators 132 and may feed into an ADC, which controller 112 may calculate back EMF from. As an alternative, a back EMF detection circuit may measure a net voltage across one or more stimulators 132 using a differential amplifier and subtracting the net voltage from a known applied voltage. For transcutaneous electrodes, an impedance measurement circuit may apply a small test current through an electrode such as one of stimulators 132 and may measure a resulting voltage. Based on data collected and/or analyzed by direct and/or indirect measurements of interchangeable stimulation assembly 120, controller 112 may calculate and/or predict an operational status and/or projected life time of interchangeable stimulation assembly 120. For instance, changes in current draw, back EMF, and/or impedance may indicate interchangeable stimulation assembly 120 may be nearing the end of its service life. In some embodiments, controller 112 may perform a diagnostic check of interchangeable stimulation unit 120. A diagnostic check may include testing one or more stimulators 132 with various voltages and/or currents, checking if firmware is up to date, checking if software is up to date, and/or identifying any of the possible metrics of interchangeable stimulation assembly 120 described above, without limitation. Controller 112 may alert a patient of an error identified by a diagnostic check. Alerts may include vibratory, audible, visual, or other alerts. For instance, controller 112 may be in communication with a light emitting device, such as a light emitting diode (LED) which may be used to communicate information to a patient. In some embodiments, controller 112 may be in communication with a speaker, such as a piezoelectric speaker, which may be used to communicate information to a patient. Information communicated to a patient by controller 112 may include one or more errors identified by one or more diagnostic or other system checks, calibration statuses, operational status, charging status, and/or other statuses or information. For instance, a yellow light of an LED in communication with controller 112 may indicate a diagnostic check is being performed by controller 112, a red light may indicate one or more errors, and a green light may indicate systems are operational. Likewise, a short beep be a speaker in communication with controller 112 may indicate a diagnostic check is being performed, rapid beeps may indicate an error was found, and three sequential short ascending beeps may indicate interchangeable stimulation assembly 120 is ready for use. Embodiments of performance metrics and comparisons thereof of interchangeable stimulation assembly 120 may be described in further detail below with reference to FIGS. 13-15.

Still referring to FIG. 1, interchangeable stimulation assembly 120 may include an identification unit, such as, identifier 136. Identifier 136 may include, but is not limited to, radio frequency identification (RFID) tags, magnetic stripes, near field communication (NFC) tags, digital watermarking, micro dots, memory chips, and/or a unique combination of resistors. Identifier 136 may provide an identity of interchangeable stimulation assembly 120, which may be a unique combination of numbers and/or characters that represent a specific interchangeable simulation assembly 120 out of a plurality of interchangeable stimulation assemblies 120. Interchangeable stimulation assembly 120 may be tracked along its lifecycle using one or more identifiers 136. In some embodiments, controller 112 may be configured to associate and/or assign a specific interchangeable stimulation assembly 120 to a specific patient based on identifier 136. In some embodiments, controller 112 may be configured to identify interchangeable stimulation assembly 120 by identifier 136. Controller 112 may be configured to generate a lifecycle for interchangeable stimulation assembly 120 based on identifier 136. For instance and without limitation, controller 112 may link one or more lifecycle metrics to identifier 136. Controller 112 may store one or more metrics of interchangeable assembly 120 along with an identification of interchangeable assembly 120 in an on-board memory. In some embodiments, controller 112 may communicate one or more metrics of interchangeable stimulation assembly 120 to an external computing device and/or with a cloud data system through a wireless communication unit of wearable device 104. Controller 112 may be configured to generate a system diagnostic result specific to a specific interchangeable stimulation assembly 120 via identifier 136. Results specific to interchangeable stimulation assembly 120 may be stored in an on-board memory of wearable device 104 and/or may be communicated to an external computing device via a wireless communication unit of wearable device 104. An individual may be able to track specific lifecycles of specific interchangeable stimulation assemblies 120 based on their identifiers 136. In some embodiments, various arrangements of one or more stimulators 132 may be specific to a specific interchangeable stimulation assembly 120. For instance a first interchangeable stimulation assembly 120 having a first identifier 136 and may have stimulators 132 including vibratory motors, while a second interchangeable stimulation assembly 120 may have a second identifier 136 and may have stimulators 132 including electrodes. Combinations of stimulators 132 may be implemented across various interchangeable stimulation assemblies 120, without limitation.

With continued reference to FIG. 1, wearable device may include a wireless communication unit. A “wireless communication unit” as used throughout this disclosure refers to a device capable of transmitting and/or receiving electromagnetic signals. Wireless communication units may include, but are not limited to, inductive coils, Wi-Fi chips, Bluetooth modules, Cellular communication devices, and/or other wireless communication units. Controller 112 may be configured to communicate with an external computing device through a wireless communication unit, such as, but not limited to, a smartphone, laptop, desktop, server, and/or other computing device. In some embodiments, controller 112 may be configured to communicate to a patient through a mobile application via a wireless communication unit of wearable device 104. Controller 112 may provide information to a patient through a mobile application, web portal, and/or other form of communication. Information provided by controller 112 to a patient may include, but is not limited to, sensor data, stimulation output 124 parameters, trends in symptom severity, lifecycle metrics of interchangeable stimulation assembly 120, connection status of interchangeable stimulation assembly 120 to electrical connector 116, and/or other data. Controller 112 may be configured to indicate to a user that interchangeable stimulation assembly 120 may be malfunctioning, have a reduced capacity, has a connection issue with electrical connector 116 and/or a communication issue with controller 112, and/or other information. In some embodiments, a patient may provide one or more commands to controller 112 through a mobile application, web portal, or other communication method. Commands may include, but are not limited to, adjustment of one or more stimulation parameters of stimulation output 124, activation of interchangeable stimulation assembly 120, deactivation of interchangeable stimulation assembly 120, disconnection of power and/or data from controller 112 to interchangeable simulation assembly 120 via electrical connector 116, and/or other commands. Controller 112 may be configured to electrically disconnect interchangeable stimulation assembly 120 from electrical connector 116 and/or power supply 108. For instance, a patient may interact with a mobile application in communication with wearable device 104 via a wireless communication unit of wearable device 104 and may provide one or more commands to controller 112, which controller 112 may perform.

In some embodiments, controller 112 may be configured to determine a projected lifecycle of interchangeable stimulation assembly 120. For instance, controller 112 may determine based on one or more metrics of interchangeable stimulation assembly 120, a replacement date of interchangeable stimulation assembly 120. Replacement dates may include periods of time such as, but not limited to, days, weeks, months, years, and the like. Controller 112 may send a push notification to a computing device of a patient, such as a smartphone, indicating a projected replacement date of interchangeable stimulation assembly 120.

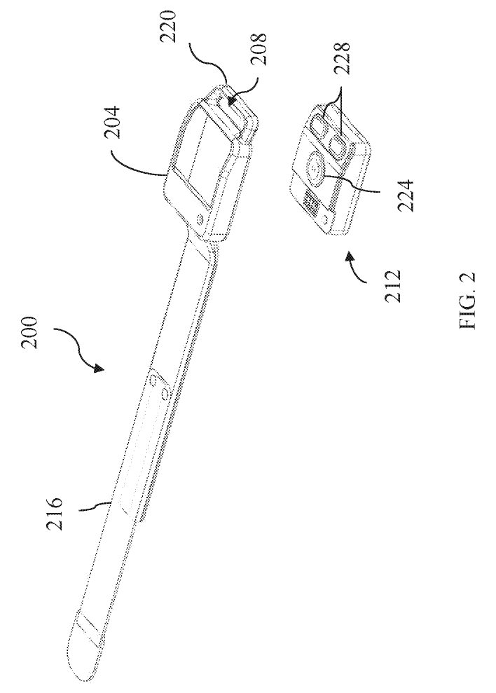

Referring now to FIG. 2, an illustration of an interchangeable stimulation assembly adjacent to a controlling unit is presented. Interchangeable stimulation assembly 200 may be as described above with reference to FIG. 1, without limitation. Interchangeable stimulation assembly 200 may include band 216 and housing 208. Housing 208 may be shaped to receive controlling unit 212. Controlling unit 212 may include controller 112, power supply 108, and/or sensor 128 as described above with reference to FIG. 1, without limitation. Controlling unit 212 may include electrical connector 228. Electrical connector 228 may be the same as electrical connector 116 as described above with reference to FIG. 1. Electrical connector 228 may include one or more pogo pins, in some embodiments. Electrical connector 228 may be positioned at a side of controlling unit 212 with respect to a center of controlling unit 212. For instance and without limitation, electrical connector 228 may be positioned at a top of controlling unit 212. Electrical connector 228 may be indented into a top side of controlling unit 212, in some embodiments. For instance and without limitation, electrical connector 228 may be positioned lower than one or more other parts of controlling unit 212. Other parts of controlling unit 212 may include, but are not limited to, mode select button 232 and adjusts 236. Mode select button 232 may be depressible, capacitive, or other variations of buttons. Mode select button 323 may allow for a patient to turn on and off various stimulation output modes of controlling unit 212. Adjusters 236 may be depressible, capacitive, or other variations of buttons. Adjusters 236 may allow a patient to adjust stimulation output of interchangeable stimulation assembly 200, in some embodiments.

In some embodiments, interchangeable stimulation assembly 200 may include band 216. Band 216 may be made out of a flexible material, such as a polymer, rubber, or other material. In some embodiments, band 216 may be made from leather. Band 216 may be operable to loop around a patient’s body part, such as a wrist, and connect at holder 220. Holder 220 may be made out of housing 208, in some embodiments. Holder 220 may have a slot 224. Slot 224 may be rectangular, triangular, circular, or other shapes. In some embodiments, slot 224 may be adapted to receive an end of band 216. Band 216 may pass through slot 220, which may form a loop around a body part of a patient. Band 216 may have one or more securing devices that may secure band 216 to holder 220. For instance, band 216 may be a magnetic strap, Velcro, or other types of securing device. Band 216 may be adjustable and may have two or more notches each of which may correspond to a certain length of band 216 which may allow band 216 to wrap around various sized body parts of a patient, such as wrist, arms, and the like.

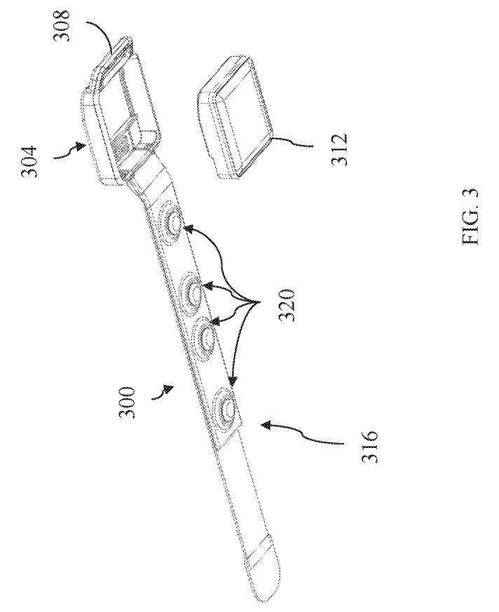

Referring now to FIG. 3, an illustration of a bottom view of interchangeable stimulation assembly 300 adjacent to controlling unit 300 is presented. Interchangeable stimulation assembly 300 may include housing 304 and/or holder 308, which may be described above with reference to FIG. 2. Interchangeable stimulation assembly 300 may include electrical connector port 312. Electrical connector port 312 may be a receiving end of an electrical connector, such as electrical connector 228 as described above with reference to FIG. 2. In some embodiments, electrical connector port 312 may include one or more receiving slots for one or more pogo pins. Electrical connector port 312 may protrude downwards from housing 304, in some embodiments. For instance an electrical connector of controlling unit 316 may be indented into a top side of controlling unit 316 and electrical connector port 312 may protrude downwards to enable a mating of controlling unit 316 and interchangeable stimulation assembly 300 via an electrical connector of controlling unit 316. Electrical connector port 312 may be positioned at a side of housing 304, such as, but not limited to, a top, left, right, or rear side of housing 304. In some embodiments, electrical connector port 312 may be positioned adjacent to band 320 of interchangeable stimulation assembly 300. An adjacent positioning of electrical connector port 312 to band 320 may allow for a more direct connection between controlling unit 312 and interchangeable stimulation assembly 300.

Interchangeable stimulation assembly 300 may include one or more stimulators 324. Stimulators 324 may be as described above with reference to FIG. 1. Stimulators 324 may be circular, triangular, rectangular, or other shapes. Stimulators 324 may be electric, thermal, vibratory, and/or ultrasonic stimulators. In some embodiments, interchangeable stimulation assembly 300 may include two or more stimulators 324. Stimulators 324 may be embedded into a portion of band 328. In some embodiments, stimulators 324 may protrude downwards from band 328. Each stimulator of stimulators 324 may be positioned to contact a specific portion of a patient’s body. For instance, two or more stimulators 324 may be positioned on band 328 of interchangeable stimulation assembly 300 to contact a proprioceptive nerve and/or tissue of a patient. In some embodiments, interchangeable stimulation assembly 300 may include sets of stimulators 324. For instance and without limitation, interchangeable stimulation assembly 300 may include a first set of stimulators 324 and a second set of stimulators 324. A first set of stimulators 324 may be two or more stimulators 324 that are positioned at distal ends of band 328. For instance and without limitation, a first stimulator 324 of a set of stimulators 324 may be positioned at a distal end of band 328 and a second stimulator 324 of a set of stimulators 324 may be positioned at a proximal end of band 328. When placed on a patient, band 328 may wrap around the patient’s body part which may positioned a first set of stimulators 324 on opposite sides of the patient’s body part, such as a wrist. An opposite positioning of two or more stimulators 324 may allow stimulation of two or more proprioceptive nerves and/or tissues of a patient’s body part. In some embodiments, interchangeable stimulation assembly 300 may include a second set of stimulators 324. A second set of stimulators 324 may include two or more stimulators 324 that may be positioned adjacent to one another. For instance and without limitation, a second set of stimulators 324 may include two or more stimulators that may be placed about 1 cm away from each other. A second set of stimulators 324 that may be positioned proximate to one another may concentrate a stimulation of a patient’s proprioceptive nerve and/or tissue. For instance and without limitation, a second set of stimulators 324 may contact an area of a patient’s body part while band 328 is wrapped around the patient’s body part.

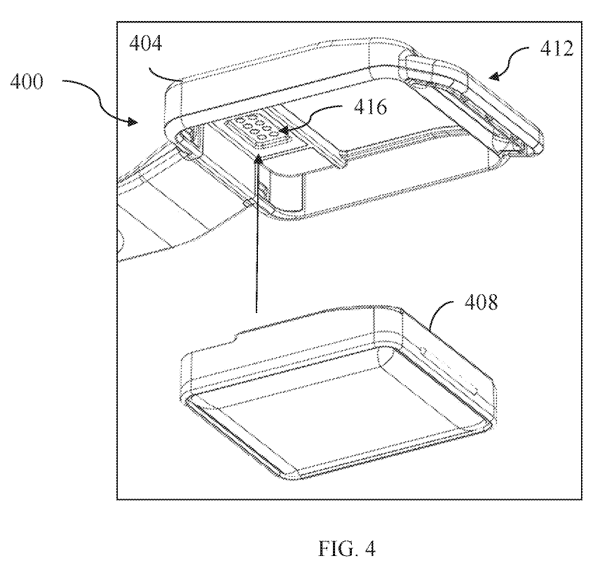

Referring now to FIG. 4, a zoomed-in illustration of an interchangeable stimulation assembly connecting to a controlling unit is presented. Interchangeable stimulation assembly 400 may include housing 404 and holder 412, which may be as described above with reference to FIG. 2. Interchangeable stimulation assembly 400 may include electrical connector port 416, which may be as described above with reference to FIG. 3. Electrical connector port 416 may be positioned within housing 404 to allow for a connection between electrical connector port 416 and an electrical connector of controlling unit 420. For instance, a patient or other individual may place controlling unit 420 inside of housing 404. Controlling unit 420 may be sized to snap into housing 404 of interchange stimulation assembly 400. For instance and without limitation, dimensions of housing 404 may allow for a securing of controlling unit 420 to an interior of housing 404. Controlling unit 420 may simultaneously be housed or otherwise secured to housing 404 of interchangeable stimulation assembly 400 while being electrically connected to electrical connector port 416 of interchangeable stimulation assembly 400.

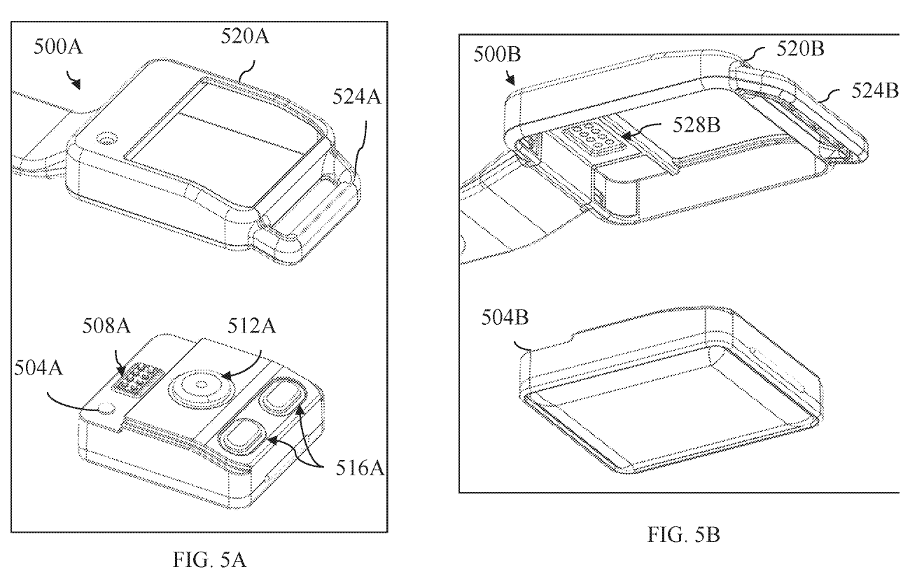

Referring now to FIG. 5A, a top view of interchangeable stimulation assembly 500A adjacent to controlling unit 504A is illustrated. Controlling unit 504A may include electrical connector 508A. Electrical connector 508A may be as described above with reference to FIG. 2. Electrical connector 508A may be indented into a side of controlling unit 504A, in some embodiments. Controlling unit 504A may include mode select button 512A and adjusters 516A, which may be as described above with reference to FIG. 3. Interchangeable stimulation assembly 500A may include housing 520A and holder 524A, which may be as described above with reference to FIG. 2.

Referring now to FIG. 5B, a bottom view of interchangeable stimulation assembly 500B adjacent to controlling unit 504B is illustrated. Interchangeable stimulation assembly 500B and controlling unit 504B may be as described above with reference to FIG. 2. Interchangeable stimulation assembly 500B may include electrical connector port 528B, which may be as described above with reference to FIG. 3.

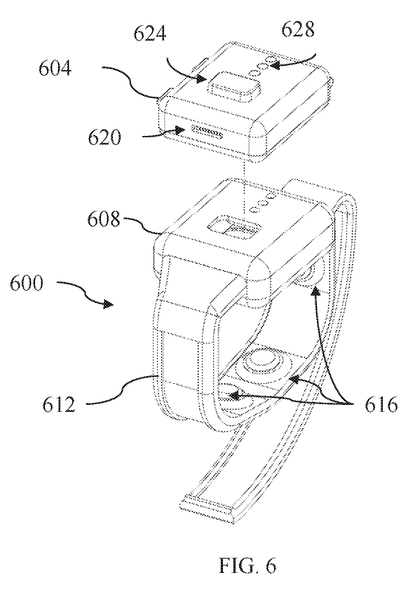

Referring now to FIG. 6, another embodiment of an interchangeable stimulation assembly 600 and controlling unit 604 is presented. Interchangeable stimulation assembly 600 may include housing 608, band 612, and stimulators 616, which may be as described above with reference to FIG. 3. Controlling unit 604 may include electrical connector port 620. Electrical connector port 620 may be a receiving end of a cable, such as, but not limited to, a flat flex cable (FFC) or other cable type. Controlling unit 604 may be adapted to be positioned within housing 608, such as in a snap-in configuration. In some embodiments, housing 608 may be flexible and may wrap around controlling unit 604.

Controlling unit 604 may include mode select button 624 and indicators 628. Mode select button 624 may be a depressible or capacitive button and may allow a patient to active and deactivate one or more stimulation outputs of controlling unit 604. In some embodiments, Indicators 628 may be light emitting diodes (LEDs) and may provide visual information to a patient. For instance in some embodiments, controlling unit 604 may include two or more indicators 628. In some embodiments, controlling unit 604 may include three indicators 628. Each indicator of indicators 628 may correspond to a specific function. For instance a first indicator 628 may correspond to a power level, a second indicator 628 may correspond to a system operation, and a third indicator 628 may correspond to a duration of stimulation. In some embodiments, indicators 628 may be adapted to emit various colors of light, such as red, blue, yellow, green, and combinations thereof. A specific color may correspond to specific indications. For instance a red color may correspond to a dead battery indication, a yellow color may correspond to an error indication, and a green color may correspond to an active stimulation. In some embodiments, indicators 628 may each correspond to a power level of controlling unit 604. For instance all three indicators 628 may emit green light which may indicate a full charge while two indicators 628 emitting green light may indicate a partial charge of controlling unit 604 and one indicator 628 emitting green light may indicate a low charge of controlling unit 604.

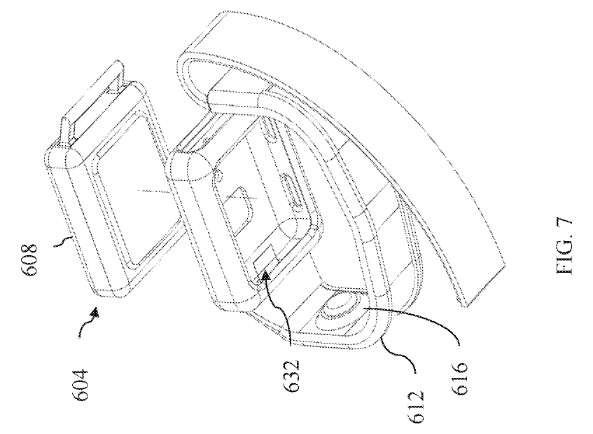

Referring now to FIG. 7, a bottom view of the interchangeable stimulation assembly and controlling unit shown in FIG. 6 is illustrated. Housing 608 of interchangeable stimulation assembly 600 may include electrical connector 632. Electrical connector 632 may be as described above with reference to FIG. 1, in some embodiments. Electrical connector 632 may be an end of a cable, such as an FFC cable. Electrical connector 632 may protrude from a side of an interior of housing 608, in some embodiments. For instance, electrical connector 632 may protrude from a side adjacent to band 612 of interchangeable stimulation assembly 600. Band 612 of interchangeable stimulation assembly 600 may house or be embedded with one or more stimulators 616, which may be as described above with reference to FIG. 3.

Referring still to FIG. 7, a patient may insert controlling unit 604 from a bottom position with respect to housing 608. In some embodiments, a bottom portion of housing 608 may be solid rather than hollow and a patient may insert controlling unit 604 from a top end of housing 608. Housing 608 may rotate or pivot to a side, such as through one or more hinges, in some embodiments. For instance and without limitation, housing 608 may open up vertically in a clockwise or counterclockwise direction. In embodiments where housing 608 may be rotatable, housing 608 may snap into a locked position such as through one or more locking slots or other locking devices.

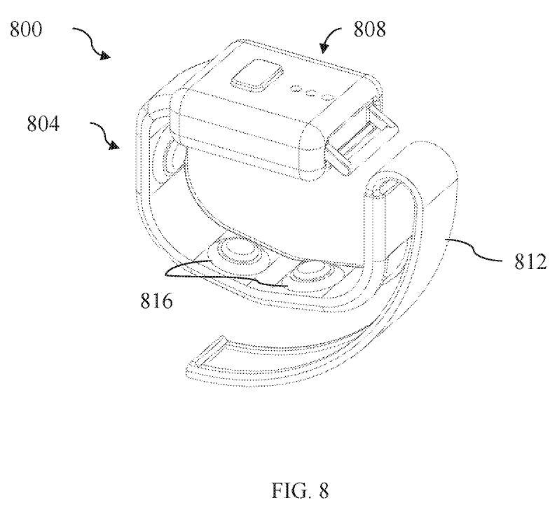

Referring now to FIG. 8, a wearable device 800 is illustrated. Wearable device 800 may include interchangeable stimulation assembly 804 and controlling unit 808. Interchangeable stimulation assembly 804 may be as described above with reference to FIGS. 1-3. Controlling unit 808 may be as described above with reference to FIGS. 1-2.

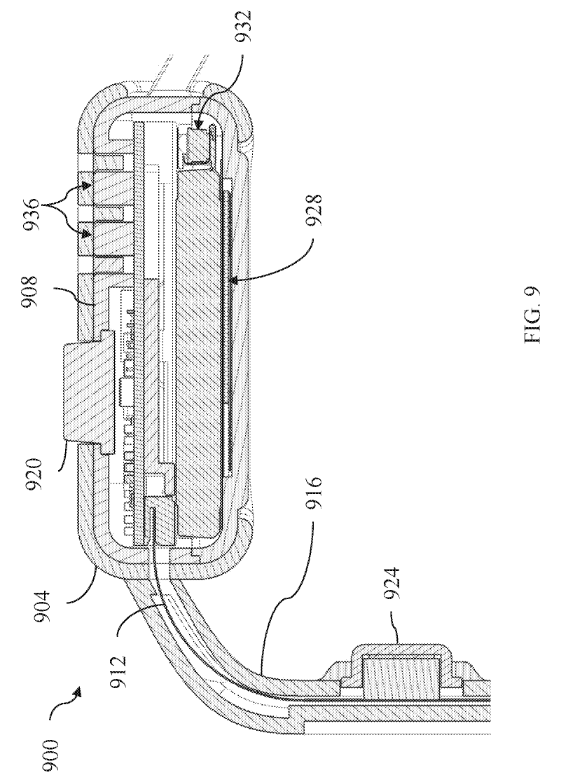

Referring now to FIG. 9, an interior view of a wearable device 900 is illustrated. Wearable device 900 may include interchangeable stimulation assembly 904 and controlling unit 908, each of which may be as described above with reference to FIGS. 1-3. Interchangeable stimulation assembly 904 may include band 912 which may house electrical connector 916. Electrical connector 916 may be a cable or other connector. In some embodiments, electrical connector 916 may connect to one or more stimulators 924 that may be embedded in band 912. Electrical connector 916 may provide a connection between controlling unit 908 and one or more stimulators 924 of band 912. In some embodiments, wearable device 900 may include wireless charging coil 928. Wireless charging coil 928 may be made of any conductive material, without limitation. Wireless charging coil 928 may include one or more loops of conductive material. In some embodiments, wireless charging coil 928 may be configured to receive wireless power via one or more magnetic fields. For instance, wireless charging coil 928 may be configured to receive power via electromagnetic induction from one or more external electromagnetic fields. Wireless charging coil 928 may provide power to battery 932. Battery 932 may be a rechargeable battery, such as a lithium-ion or other battery. In some embodiments, wearable device 900 may include light pipes 936. Light pipes 936 may be light emitting diodes, in an embodiment. Light pipes 936 may be configured to emit light and may be the same as indicators described above with reference to FIG. 6, without limitation.

Controlling unit 908 may include control circuitry 928. Control circuitry 928 may include one or more processors, capacitors, transistors, resistors, operational amplifiers, and/or other electronic components. Control circuitry 928 may be configured to regulate power delivered to one or more stimulators 924. In some embodiments, control circuitry 928 may be configured to control one or more stimulators 924. Control circuitry 928 may include a sensing circuit. A sensing circuit may be configured to sense voltages, currents, and the like of one or more stimulators 924. Control circuitry 928 may be configured to identify decreasing performance of one or more stimulators 924, such as described above with reference to FIG. 1.

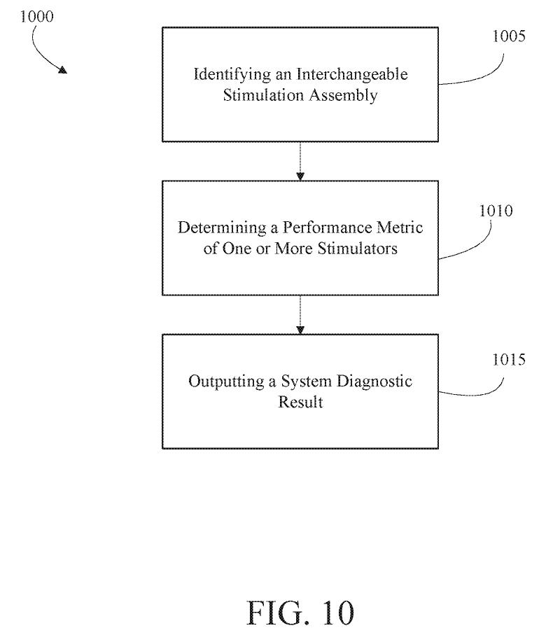

Referring now to FIG. 10, a method 1000 of tracking performance of a wearable device is presented. At step 1005, method 1000 includes identifying an interchangeable stimulation assembly. An interchangeable stimulation assembly may be identified by a controlling unit in electrical communication with the interchangeable stimulation assembly via an identifier of the interchangeable stimulation assembly. Identification of an interchangeable stimulation assembly may include producing an identity of the interchangeable simulation assembly, which may be a unique combination of numbers and/or characters specific to the interchangeable stimulation assembly. This step may be implemented as described above with reference to FIGS. 1-9, without limitation.

At step 1010, method 1000 includes determining a performance metric of one or more stimulators of an interchangeable stimulation assembly. One or more stimulators of an interchangeable stimulation assembly may be positioned along or within the interchangeable stimulation assembly to provide a stimulation output generated by a controlling unit in electrical connection with the interchangeable stimulation assembly to a body part of a patient. One or more stimulators may be electric, vibratory, ultrasonic, thermal, or other types of stimulators, without limitation. A performance metric may include, but is not limited to, peak power output values of one or more stimulators, current and/or voltage values of one or more stimulators, back EMF of one or more stimulators, and/or other parameters described throughout this disclosure, without limitation. This step may be implemented as described above with reference to FIGS. 1-9, without limitation.

At step 1015, method 1000 includes outputting a system diagnostic result. A system diagnostic result may include an indication that one or more stimulators of an interchangeable stimulation assembly are malfunctioning. In some embodiments, a system diagnostic result may include an indication that a patient should replace an interchangeable stimulation assembly. A system diagnostic result may be output via audio or visual interfaces of a wearable device, such as LEDs, piezoelectric speakers, and/or other devices. In some embodiments, a system diagnostic result may be communicated to a computing device of a patient, such as through a mobile application of smartphone. This step may be implemented as described above with reference to FIGS. 1-9, without limitation.

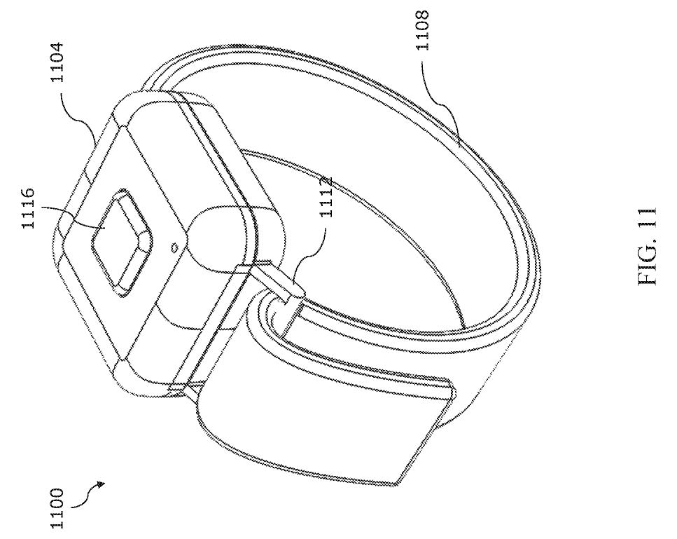

Referring now to FIG. 11, an illustration of a wearable device 1100 is presented. In some embodiments, the wearable device 1100 may include a housing 1104 that may be configured to house one or more components of the wearable device 1100. For instance, the housing 1104 of the wearable device 1100 may include a circular, ovular, rectangular, square, or other shaped material. In some embodiments, the housing 1104 may have a length of about 5inches, a length of about 5 inches, and a width of about 5 inches, without limitation. In some embodiments, the housing 1104 may have a length of about 1.5 inches, a width of about 1.5inches and a height of about 0.5 inches. The housing 1104 of the wearable device 1100 may have an interior and an exterior. An interior of the housing 1104 of the wearable device 1100 may include, but is not limited to, one or more sensors, transducers, energy sources, processors, memories, and the like, such as those described above with reference to FIG. 1. In some embodiments, an exterior of the housing 1104 of the wearable device 1100 may include one or more interactive elements 1116. An “interactive element” as used in this disclosure is a component that is configured to be responsive to user input. The interactive element 1116 may include, but is not limited to, buttons, switches, and the like. In some embodiments the wearable device 1100 may have a singular interactive element 1116. In other embodiments, the wearable device 1100 may have two or more interactive elements 1116. In embodiments where the wearable device 1100 has a plurality of interactive elements 1116, each interactive element 1116 may correspond to a different function. For instance, a first interactive element 1116 may correspond to a power function, a second interactive element 1116 may correspond to a waveform adjustment, a third interactive element 1116 may correspond to a mode of the wearable device 1100, and the like. In some embodiments, the wearable device 1100 may include a touch screen display.

In some embodiments, the wearable device 1100 may include one or more batteries. For instance, and without limitation, the wearable device 1100 may include one or more replaceable batteries, such as lead-acid, nickel-cadmium, nickel-metal hydride, lithium-ion, and/or other battery types. The housing 1104 of the wearable device 1100 may include a charging port that may allow access to a rechargeable battery of the wearable device 1100. For instance and without limitation, the wearable device 1100 may include one or more rechargeable lithium-ion battery and a charging port of the housing 1104 of the wearable device 1100 may be a USB-C, micro-USB, and/or other type of port. A battery of the wearable device 1100 may be configured to charge at a rate of about 10W/hr. A battery of the wearable device 1100 may be configured to charge at about 3.7V with a current draw of about 630mA. A battery of the wearable device 1100 may have a capacity of about 2.5Wh, greater than 2.5Wh, or less than 2.5Wh, without limitation. In some embodiments, the wearable device 1100 may include one or more wireless charging circuits that may be configured to receive power via electromagnetic waves. The wearable device 1100 may be configured to be charged wirelessly at a rate of about 5W/hr through a charging pad or other wireless power transmission system. In some embodiments, a battery of the wearable device 1100 may be configured to be charged at about 460mA, greater than 460mA, or less than 460mA.

Still referring to FIG. 11, the wearable device 1100 may include an attachment system. An attachment system may include any component configured to secure two or more elements together. For instance, and without limitation, the wearable device 1100 may include a wristband 1108. The wristband 1108 may include one or more layers of a material. For instance and without limitation, the wristband 1108 may include multiple layers of a polymer, such as rubber. The wristband 1108 may have an interior and an exterior. An interior and an exterior of the wristband 1108 may be a same material, texture, and the like. In other embodiments, an interior of the wristband 1108 may be softer and/or smoother than an exterior of the wristband 1108. As a non-limiting example, an interior of the wristband 1108 may be a smooth rubber material while an exterior of the wristband 1108 may be a Velcro material. The wristband 1108 may have a thickness of about 2 mm. In other embodiments, the wristband 1108 may have a thickness of greater than or less than about 2mm. The wristband 1108 may be a rubber band, Velcro strap, and the like. In some embodiments, the wristband 1108 may be adjustable. For instance, the wristband 1108 may be a flexible loop that may self-attach through a Velcro attachment system. In some embodiments, the wristband 1108 may attach to one or more hooks 1112 of an exterior of the housing 1104 of the wearable device 1100. In some embodiments, the wristband 1108 may be magnetic. In other embodiments, the wristband 1108 may include a column, grid, or other arrangement of holes that may receive a latching from the hook 1112.

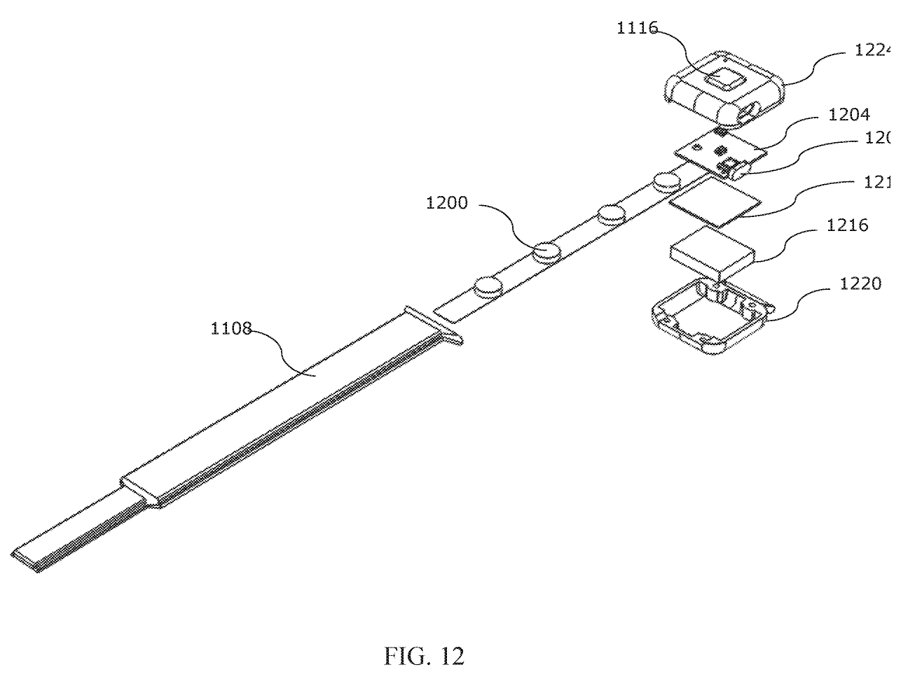

Referring now to FIG. 12, an exploded side view of the wearable device 1100 is shown. The wearable device may include mechanical transducers 1200. The mechanical transducers 1000 may be housed within the wristband 1108. The wristband 1108 may be configured to interface with a user’s writs. The wearable device may have a top half of a housing 1224 and a bottom half of a housing 1220. In some embodiments, between the top half 1224 and the bottom half 1220, a printed circuit board 1204 (PCB) may be positioned. Further, a silicone square may be positioned to insulate a bottom of the PCB 43, which may be positioned above a battery 1216. The battery 1216 may include protection circuitry to protect from overcharging and unwanted discharging. In some embodiments, the wearable device 1100 may include a magnetic connector 1208. The magnetic connector 1208 may be configured to align the wearable device with a charging pad, station, and the like. The magnetic connector 1208 may be configured to receive power wirelessly to recharge the battery 1216. The magnetic connector 1208 may be coupled to the battery 1216 and mounted in the housing 1220 and/or 1224. In some embodiments, the magnetic connector 1208 may be inserted into the PCB 1204. The magnetic connector 1208 may be configured to mate with a connector from an external charger.

Referring now to FIG. 13, a flowchart illustrating a process 1300 for performing a system diagnostic check is presented. At step 1305, process 1300 includes performing normal wearable device and interchangeable stimulation assembly functions. For instance, a wearable device may be worn by a patient and my generate and output a stimulation output to one or more body parts of a patient over a period of time. This step may be implemented as described above without limitation in FIGS. 1-12.

At step 1310, process 1300 includes triggering a diagnostic trigger of an interchangeable stimulation assembly. A diagnostic trigger may be triggered by a controlling unit in electrical communication with an interchangeable stimulation assembly. A diagnostic trigger may include a voltage, current, power, frequency, or other measurement of one or more stimulators of an interchangeable stimulation assembly. For instance, one or more stimulators of an interchangeable stimulation assembly may produce current, voltage, power, frequency, and/or other outputs that may be above or below a threshold value. A threshold value may be determine by a controlling unit. In other embodiments, a threshold value may be entered to a memory of a controlling unit. In some embodiments, a diagnostic trigger may be manually performed. For instance, a patient may interact with one or more interfaces of a controlling unit to cause the controlling unit to enter a system diagnostic check mode. In some embodiments, a controlling unit may be triggered to perform a system diagnostic check at certain time intervals, such as, but not limited to, every 12 hours, every 24 hours, every 48 hours, and so on.

At step 1315, process 1300 includes running an interchangeable stimulation assembly diagnostic procedure. A diagnostic procedure may include testing one or more stimulators of an interchangeable stimulation assembly through a sensing circuit or other sensing device of a controlling unit. A sensing circuit or other sensing device of a controlling unit may output on current, voltage, back EMF, or other values that may indicate one or more stimulators are malfunctioning. In some embodiments, a diagnostic procedure may include checking a firmware of one or more stimulators of an interchangeable stimulation assembly. A firmware may be checked for a most recent firmware version and/or to identify and errors that may have occurred during operation or outside operation of an interchangeable stimulation assembly. Errors may include, but are not limited to, activation of only of a plurality of stimulators, high power consumption of one or more stimulators, asynchronization of one or more stimulators while providing stimulation output, and/or other errors.

At step 1320, process 1300 includes checking for a stimulator failure of an interchangeable stimulation assembly. A stimulator failure may include, but is not limited to, outdate firmware of an interchangeable stimulation assembly, inoperability of a stimulator, current and/or voltage values that fall above or below a safe operation threshold, and/or other failures such as those described above at step 1315.

At step 1325, if a failure of one or more stimulators is detected, a controlling unit may lock activation of one or more stimulators of an interchangeable stimulation assembly. Locking of activation of one or more stimulators may include preventing voltages and/or currents from being provided to one or more stimulators of an interchangeable stimulation assembly. In some embodiments, a controlling unit may prompt a patient to replace an interchangeable stimulation assembly. Prompts may include, but are not limited to, visual indicators such as LEDs of a controlling unit, audio indicators such as piezoelectric or other speakers of a controlling unit, and/or prompting a patient through an external computing device, such as an application of a smartphone, laptop, and the like which a controlling unit may be in communication of via a communication module of the controlling unit.

At step 1330, if no stimulator failure is detected, process 1300 may repeat back to step 1305 and process 1300 may start over indefinitely.

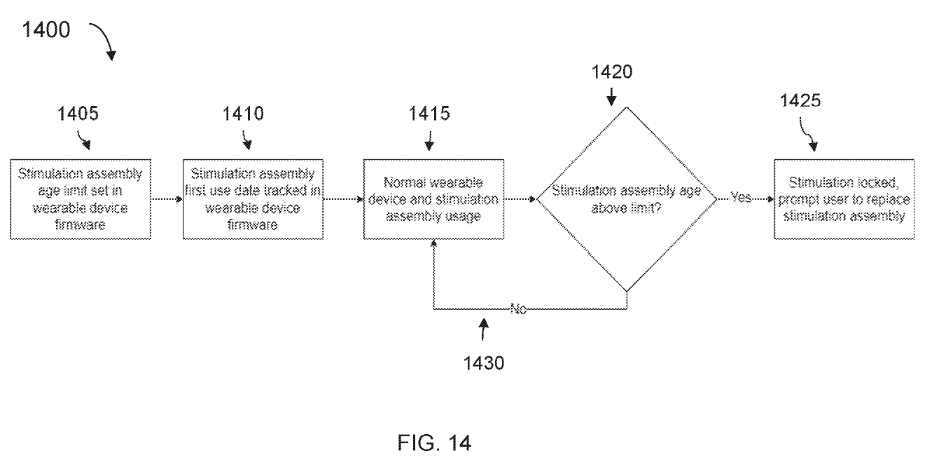

Referring now to FIG. 14, an embodiment of a process 1400 for checking an age of one or more stimulators of an interchangeable stimulation assembly is illustrated. At step 1405, process 1400 includes setting an age limit of an interchangeable stimulation assembly in a firmware of the interchangeable stimulation assembly and/or a controlling unit. An age limit may include any period of time, without limitation. For instance an age limit may be about 3 months, about 6 months, about 12 months, about 24 months, greater than 24 months, or less than 3 months. An age of one or more stimulators of an interchangeable stimulation assembly may be determined based on historical operation data of a plurality of interchangeable stimulation assemblies. In some embodiments, an age of one or more stimulators of an interchangeable stimulation assembly may correspond to a performance threshold of one or more stimulators. As a non-limiting example, about 6 months of average operation of one or more stimulators may reduce a peak power output of the one or more stimulators by about 80%.

At step 1405, process 1400 includes checking a first use date of an interchangeable stimulation assembly via a controlling unit. A first use date may be an initial date of operation of an interchangeable stimulation assembly. For instance and without limitation, a first use date may be a first day a patient activated one or more stimulators of an interchangeable stimulation assembly. A controlling unit in electrical connection with an interchangeable stimulation assembly may check a first use date of the interchangeable stimulation assembly through firmware of the interchangeable stimulation assembly. Firmware of an interchangeable stimulation assembly may provide dates, times, locations, and/or other information of an initial activation of one or more stimulators of the interchangeable stimulation assembly.

At step 1415, normal processes and operation of a wearable device including a controlling unit and an interchangeable stimulation assembly occur. This step may be implemented as described above, without limitation, in FIGS. 1-13.

At step 1420, an interchangeable stimulation assembly age is compared to an age threshold. An age threshold may be a range of periods of time from an initial start date of an interchangeable stimulation assembly that may, if surpassed, indicate the interchangeable stimulation assembly should be replaced with a new interchangeable stimulation assembly. An age threshold may be determined by a controlling unit based on one or more factors, such as, but not limited to, daily usage time, type of stimulator, average frequencies used in stimulation output, length of high power operations, and/or other factors that may be determined by the controlling unit. An age threshold may be specific to a specific interchangeable stimulation assembly on a per device basis, based on one or more factors as described previously. In other embodiments, a same age threshold may be used for two or more interchangeable stimulation assemblies. A controlling unit may compare a number of days, weeks, months, and the like to an age threshold. An age threshold may be a single value, such as about 6 months. In other embodiments, an age threshold may be within a range of values which may include any range of periods of time without limitation. For instance and without limitation, an age threshold may be about 4 months to about 6 months, at which a controlling unit may compare a period of time from an initial start date of an interchangeable stimulation assembly. A controlling unit may prompt a patient that an interchangeable stimulation assembly may be approaching a recommended replacement date, in some embodiments. A controlling unit may compare an age threshold with one or more other performance metrics of an interchangeable stimulation assembly and may shorten or lengthen an age threshold based on how the one or more other performance metrics. For instance, a peak power output of an interchangeable stimulation assembly may be within an operational range despite the interchangeable stimulation assembly reaching an age threshold, to which a controlling unit may add additional days, weeks, months, or other periods of time to the age threshold.

At step 1425, if an age of an interchangeable stimulation assembly surpasses a threshold age value, a controlling unit may lock activation of one or more stimulators of the interchangeable stimulation assembly. A controlling unit may prompt a patient to replace an interchangeable stimulation assembly. A prompt may include an audio or visual prompt that may be provided through one or more interfaces of a controlling unit, such as LEDs or piezoelectric speakers, and/or may be provided to an external computing device via a communication module of a controlling unit.

At step 1430, if an age of an interchangeable stimulation assembly has not surpassed an age threshold, process 1300 may revert back to step 1315 and normal operations may occur.

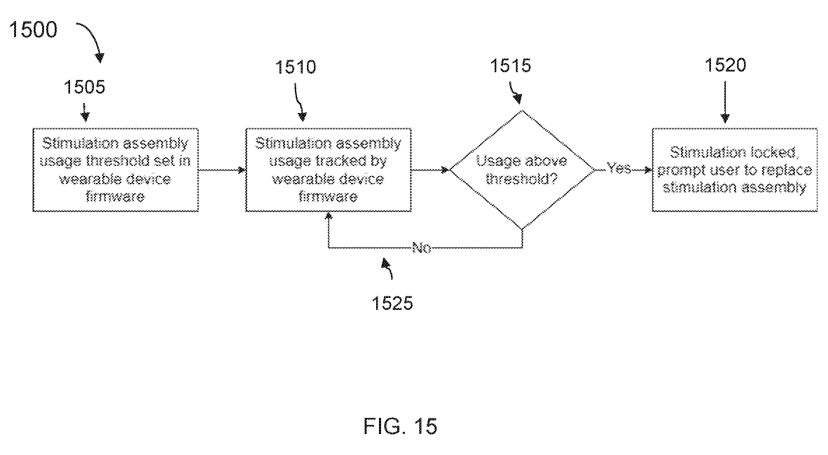

Referring now to FIG. 15, a flow diagram of a process 1500 of determining wear of an interchangeable stimulation assembly is presented. At step 1505, a usage threshold may be set in a firmware of an interchangeable stimulation assembly. A usage threshold may include one or more cycles of simulation output, total power output, and/or other values that may be associated with operation of one or more stimulators of an interchangeable stimulation assembly. A usage threshold may be set via user input or other inputs to a firmware of an interchangeable stimulation assembly.

At step 1510, process 1500 includes tracking a usage of an interchangeable stimulation assembly. A usage of an interchangeable stimulation assembly may be tracked by a controlling unit in communication with the interchangeable stimulation assembly. For instance, a controlling unit may interact with a firmware of an interchangeable stimulation assembly. A firmware of an interchangeable stimulation assembly may include data such as, but not limited to, initial start date, number of stimulation output cycles generated, total power output, and/or other information. A controlling unit may continually check for usage metrics of an interchangeable stimulation assembly. In other embodiments, a controlling unit may periodically check for usage metrics of an interchangeable stimulation assembly. For instance, a controlling unit may check for usage metrics of an interchangeable stimulation assembly over a period of 12 hours, 24 hours, 48 hours, less than 12 hours, or greater than 48 hours, without limitation.

At step 1515, process 1500 includes checking if a usage of an interchangeable stimulation assembly is above a usage threshold value. A usage threshold value may be as described above at step 1505. A controlling unit may check one or more usage metrics of an interchangeable stimulation assembly with a usage threshold value via firmware of the interchangeable stimulation assembly.

At step 1520, if one or more usage metrics of an interchangeable stimulation assembly surpass a usage threshold value, stimulation of one or more stimulators of the interchangeable stimulation assembly may be locked.

At step 1525, if one or more usage metrics of an interchangeable stimulation assembly are below a usage threshold value, process 1500 may recert back to step 1510.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. An assembly for a wearable neurostimulation device, comprising:
a controlling unit comprising a processor, a sensor in communication with the processor, and a power supply in communication with the sensor and the processor; and
an interchangeable stimulation assembly comprising:
an attachment system adapted to securely attach to the controlling unit;
an electrical connector configured to electrically connect to the controlling unit;
a band configured to be worn by a body part of a patient and comprising one or more stimulators in communication with the electrical connector; and
a sensing circuit in communication with the controlling unit, wherein the controlling unit is configured to:
receive sensor data from the sensor;
generate a stimulation output based on the sensor data;
command the one or more stimulators to stimulate a portion of the body part of the patient with the stimulation output; and
track a lifecycle metric of the one or more stimulators based on data from the sensing circuit.

2. The assembly of claim 1, wherein the controlling unit is further configured to lock, based on the lifecycle metric, activation of the one or more stimulators of the interchangeable stimulation assembly.

3. The assembly of claim 2, wherein the controlling unit is further configured to prompt the patient to replace the interchangeable stimulation assembly through one of a visual or audial indicator.

4. The assembly of claim 1, wherein the controlling unit is further configured to:
identify a performance metric of each stimulator of the one or more stimulators based on data from the sensing circuit; and
output a system diagnostic result of each stimulator of the one or more stimulators.

5. The assembly of claim 1, wherein the controlling unit is further configured to:
identify a first use date of the interchangeable stimulation assembly;
compare the first use date to a usage threshold; and
based on the comparison, output a system diagnostic result of the interchangeable stimulation assembly.

6. The assembly of claim 5, wherein the controlling unit is further configured to lock activation of the one or more stimulators of the interchangeable stimulation assembly based on the system diagnostic result.

7. The assembly of claim 1, wherein the interchangeable stimulation assembly further comprises an identification unit, and wherein the controlling unit is configured to identify the interchangeable stimulation assembly via the identification unit and to associate the lifecycle metric with an identity of the interchangeable stimulation assembly.

8. The assembly of claim 1, wherein the controlling unit is further configured to determine an estimated lifetime of the interchangeable stimulation assembly based on the lifecycle metric.

9. The assembly of claim 1, wherein the one or more stimulators are positioned to provide the stimulation output to a proprioceptive nerve in a proprioceptive tissue of one of flexor carpi radialis, flexor carpi ulnaris, extensor carpi radialis, extensor carpi ulnaris, or a combination thereof of the patient.

10. The assembly of claim 1, wherein the electrical connector is operable to provide both a power connection between the power supply and the one or more stimulators and a data connection between the controlling unit and the one or more stimulators.

11. A method of tracking performance of an interchangeable stimulation assembly for a wearable neurostimulation device, the method comprising:
electrically connecting, via an electrical connector of the interchangeable stimulation assembly, a controlling unit to the interchangeable stimulation assembly;
receiving, at the controlling unit, sensor data from a sensor;
generating, at the controlling unit, a stimulation output based on the sensor data;
commanding, by the controlling unit, one or more stimulators to stimulate a portion of a body part of a patient with the stimulation output; and
tracking, by the controlling unit, a lifecycle metric of the one or more stimulators based on data from a sensing circuit in communication with the controlling unit.

12. The method of claim 11, further comprising locking, by the controlling unit based on the lifecycle metric, activation of the one or more stimulators of the interchangeable stimulation assembly.

13. The method of claim 12, further comprising prompting the patient to replace the interchangeable stimulation assembly through one of a visual or audible indicator.

14. The method of claim 11, wherein tracking the lifecycle metric further comprises: identifying a performance metric of each stimulator of the one or more stimulators based on data from the sensing circuit; and
outputting a system diagnostic result of each stimulator of the one or more stimulators.

15. The method of claim 11, further comprising:
identifying, by the controlling unit, a first use date of the interchangeable stimulation assembly;
comparing the first use date to a usage threshold; and
based on the comparison, outputting a system diagnostic result of the interchangeable stimulation assembly.

16. The method of claim 15, further comprising locking, by the controlling unit, activation of the one or more stimulators of the interchangeable stimulation assembly based on the system diagnostic result.

17. The method of claim 11, further comprising:
identifying, by the controlling unit, the interchangeable stimulation assembly via an identification unit of the interchangeable stimulation assembly; and
associating the lifecycle metric with an identity of the interchangeable stimulation assembly.

18. The method of claim 11, further comprising determining, by the controlling unit, an estimated lifetime of the interchangeable stimulation assembly based on the lifecycle metric.

19. The method of claim 11, wherein the one or more stimulators are positioned to provide the stimulation output to a proprioceptive nerve in a proprioceptive tissue of one of flexor carpi radialis, flexor carpi ulnaris, extensor carpi radialis, extensor carpi ulnaris, or a combination thereof of the patient.

20. The method of claim 11, wherein the electrical connector is operable to provide both a power connection between a power supply and the one or more stimulators and a data connection between the controlling unit and the one or more stimulators.
